(19) United States
(12) Patent Application Publication
(10) Pub. No.: US 2023/0330308 A1
(43) Pub. Date: Oct. 19, 2023

(54) Articles, compositions, and methods for sutureless implants

(71) Applicant: THE SECANT GROUP, LLC, Telford, PA (US)

(72) Inventors: Peter D. GABRIELE, Frisco, TX (US); Jeremy J. HARRIS, Doylestown, PA (US); Stephanie REED, Conshohocken, PA (US)

(21) Appl. No.: 18/445,110

(22) Filed: Apr. 11, 2023

Related U.S. Application Data
(60) Provisional application No. 63/362,766, filed on Apr. 11, 2022.
(60) Provisional application No. 63/362,762, filed on Apr. 11, 2022.

(51) Int. Cl. A61L 31/14; A61L 31/02; A61L 31/08; A61L 31/06

(52) U.S. Cl. CPC A61L 31/14; A61L 31/022; A61L 31/08; A61L 31/06

(57) ABSTRACT

A first process includes suturelessly fixing a surface-modified polyester textile to Nitinol wire. The surface-modified polyester textile includes a textile of a polyester having a modified surface that provides surface crosslinkable groups. A first composition includes a Nitinol layer, a passivation layer bound to the Nitinol layer, a surface-modified polyester layer, and a tie layer binding the surface-modified polyester layer to the passivation layer. A first implantable device includes a Nitinol wire, a passivation layer bound to the Nitinol wire, a surface-modified polyester textile, and a tie layer binding the surface-modified polyester textile to the passivation layer such that the surface-modified polyester textile is suturelessly fixed to the Nitinol wire. A second implantable device and a second composition include a polymer of glycerol and sebacic acid including a catechol group. A second process includes forming a polymer of glycerol and sebacic acid including a catechol group.

### CROSS-REFERENCE TO RELATED APPLICATIONS

This application claims priority to and the benefit of U.S. Provisional Application No. 63/362,762 and U.S. Provisional Application No. 63/362,766, both filed Apr. 11, 2022, each of which is hereby incorporated by reference in its entirety.

### FIELD OF THE INVENTION

The present disclosure is generally directed to articles, compositions, and methods for atramautic sutureless fixation of implantable devices, such as, for example, implantable lumens, such as, for example, stent grafts. More specifically, the present disclosure is directed to passivation of nickel titanium (Nitinol) and surface modification of implantable polyesters for fusion to Nitinol for atramautic sutureless fixation of Nitinol and atramautic sutureless fixation of implantable devices incorporating catechol-modified poly(glycerol sebacate).

### BACKGROUND OF THE INVENTION

Conventional implants, such as stent grafts, may cause trauma. For example, polyethylene terephthalate (PET) and other polyester textiles used in structural heart and other implantable polymer textile technologies are conventionally fixed to Nitinol scaffold structures with non-degradable ligatures or sutures. These ligatures form knotted features that cause traumatic contact abrasion injury to the abluminal wall of the subject vessel. Conventional aortic implants may cause aortic wall injury (AWI), which may include trauma, scratches, abrasion, endoleakage, aortic wall dissection, aneurysms, infection, abluminal intimal hyperplasia, fibrosis, wall stiffening, inflammation, disruption of hemodynamic flow, and/or nickel or other metal leaching.

Although Nitinol wire is the conventional scaffold material in stent graft construction, Nitinol is known to be corrosive in human blood. Nitinol is a metal alloy of approximately equal atomic amounts of nickel and titanium and is known to leach nickel within the first days after human implantation. Human blood chemistry, and specifically human blood pH and compositional salts, is a factor in the corrosiveness of Nitinol. Additionally, nickel metal is cytotoxic and promotes sensitization by haptenization, allowing nickel and other metals to bind major histocompatibility complex (MHC) Class II receptors that are assessed by antigen-presenting cells (APC) as “non-friendly”.

The resulting autoimmune response includes inflammation and initiation of immune innate and adaptive attack mechanisms. FIG. 1 schematically shows the role of antigen-presenting cells 10 in interacting with nickel from a Nitinol-containing implant 12 inn such mechanisms. FIG. 1 also shows the involvement of T-helper cells 14, macrophages 16, and neutrophils 18 and some subcellular components in these mechanisms and their effect on nearby endothelial cells 20.

Reducing or downregulating the innate inflammatory process in the first few days after implantation would allow the adaptive system to accommodate the implant as a new foreign body.

Costa et al. (“Mussel-Inspired Catechol Functionalisation as a Strategy to Enhance Biomaterial Adhesion: A Systematic Review”, Polymers, Vol. 13, No. 3317, 2021, 34 pages) recently reviewed catechol functionalization of polymers for adhesion applications. Costa notes that these polymers are primarily either polysaccharides or proteins, where swelling and modulus are limiting features of the material in the presence of water.

### BRIEF DESCRIPTION OF THE INVENTION

There is a need for implantable devices including implantable lumens including stent grafts that are sutureless and that are less traumatic than conventional implantable devices.

In some embodiments, a process includes suturelessly fixing a surface-modified polyester textile to Nitinol wire. The surface-modified polyester textile includes a textile of a polyester having a modified surface that provides surface crosslinkable groups.

In some embodiments, a composition includes a Nitinol layer, a passivation layer bound to the Nitinol layer, a surface-modified polyester layer, and a tie layer binding the surface-modified polyester layer to the passivation layer. The surface-modified polyester textile includes a textile of a polyester having a modified surface that provides surface crosslinkable groups.

In some embodiments, an implantable device includes a Nitinol wire, a passivation layer bound to the Nitinol wire, a surface-modified polyester textile, and a tie layer binding the surface-modified polyester textile to the passivation layer such that the surface-modified polyester textile is suturelessly fixed to the Nitinol wire. The surface-modified polyester textile includes a textile of a polyester having a modified surface that provides surface crosslinkable groups.

In some embodiments, an implantable device includes a polymer of glycerol and sebacic acid. The polymer of glycerol and sebacic acid includes a catechol group.

In some embodiments, a composition includes a polymer of glycerol and sebacic acid. The polymer of glycerol and sebacic acid includes a catechol group.

In some embodiments, a process includes forming a polymer of glycerol and sebacic acid. The polymer of glycerol and sebacic acid includes a catechol group.

### BRIEF DESCRIPTION OF THE DRAWINGS

FIG. 1 schematically shows an autoimmune response including inflammation and initiation of immune innate and adaptive attack mechanisms.

FIG. 2 schematically shows a process of modifying a polyester textile surface in an embodiment of the present disclosure.

FIG. 3 schematically shows a surface-modified polyethylene terephthalate (PET) with surface hydroxy or carboxyl groups.

FIG. 4 schematically shows a resulting surface-modified PET fiber.

FIG. 5 schematically shows a surface-modified PET sheath core.

FIG. 6 schematically shows a thermally-activated adhesive in an embodiment of the present disclosure.

FIG. 7 schematically shows a catechol-modified PGS with the catechol groups being pendant.

FIG. 8 schematically shows three catechols interacting with a titanium dioxide surface.

FIG. 9 schematically shows the potential surface features provided to PGS by catechol functionality.

FIG. 10 schematically shows catechol-modified PGS passivation layer on a sutureless implantable device.

FIG. 11 shows surface assay results for surface-treated PET, with higher absorbance indicating higher levels of surface carboxylic acid groups.

FIG. 12 shows Fourier-transform infrared (FTIR) spectroscopy data for treated PET surfaces.

FIG. 13 shows TBO assay results for treated PET surfaces, with higher absorbance indicating higher levels of surface carboxylic acid groups.

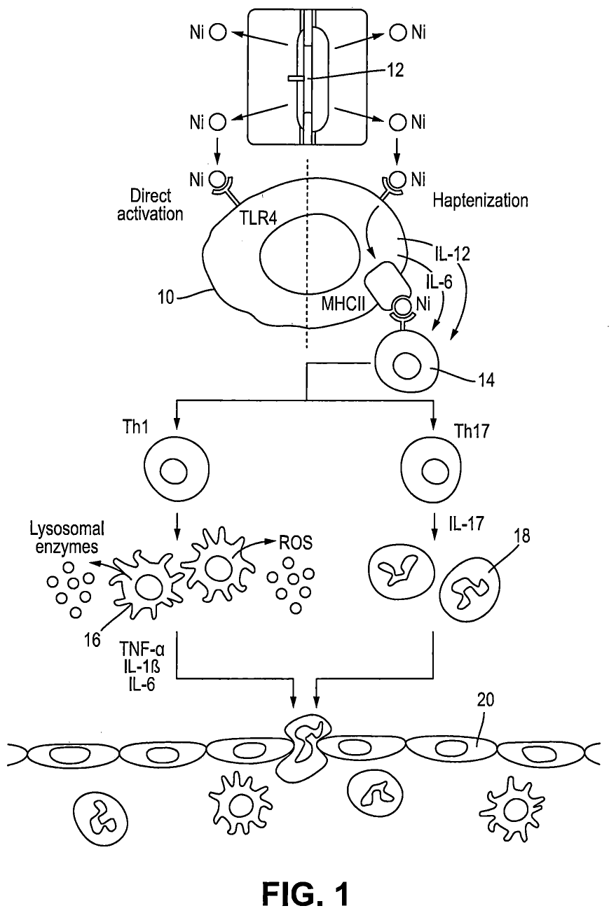

### DETAILED DESCRIPTION OF THE INVENTION

Provided are processes, compositions, and implantable devices permitting sutureless fixation of a polyester textile to Nitinol and/or including a polymer of glycerol and sebacic acid including at least one catechol group.

Embodiments of the present disclosure, for example, in comparison to concepts failing to include one or more of the features disclosed herein, surface-treat polyethylene terephthalate (PET), passivate Nitinol, fuse PET to Nitinol, provide atraumatic sutureless fixation, reduce the economic burden of manual suturing, reduce the extent of abluminal trauma and aortic wall injury resulting from the abrasion and scuffing of stent-graft sutures with the endothelial abluminal surface during implant deployment and in service, extend the service life of an aortic stent by reducing the body's natural response to foreign materials and trauma, sequester nickel and other metals leaching from Nitinol or stainless steel scaffolding, or combinations thereof.

It will be understood to those skilled in the art of adhesion science and technology that the following words or phrases, as used herein, may directly or indirectly imply the tendency of similar and or dissimilar interfacial surfaces to reversibly or irreversibly adhere or cohere to one another, less surface energy differences between surfaces and or at their respective interfacial surface boundaries, resulting in a reversible or permanent stable interface co-location: couple, coupling agent; tie, tie-layer; fix, fixation; bind, binder; fuse, fusion; modify, change; stick; or adhere. Forces acting at these interfacial boundaries may be intermolecular and chemically attractive, chemically reactive crosslinking or ionically attractive, dispersive, diffusive and or mechanical. Likewise, the term bonding may imply a chemical, electrostatic, physical-structural, or mechanical apposition. It should also be noted that in the case of one interface, co-mingling or co-penetrating the intermingling of these boundaries between surfaces may be considered an interphase. Such may be the case of a pressure sensitive adhesive (PSA) with a textile.

Efforts to create a sutureless fixation may include the development of a fabric-reactable or convertible adhesive, coating, or other interfacial process of “curing” or binding composite constructs of textile devices. PET, a conventional polyester textile surface, however, lacks the surface chemical functionality, such as hydroxy or carboxyl groups, to act as cross-link sites as shown in Formula (1):

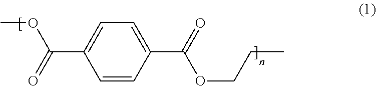

In exemplary embodiments, a process substitutes chemical bonding for suture ligations to fix in place Nitinol scaffold supports with respect to polyester graft textiles. Other polyesters appropriate for graft textiles may include, but are not limited to, polylactic acid (PLA), polyglycolic acid (PGA), poly(lactic-co-glycolic acid) (PLGA), polycaprolactone (PCL), or polyhydroxyalkanoate (PHA). Other polymers appropriate for graft textiles may include, but are not limited to, polyethylene or polypropylene.

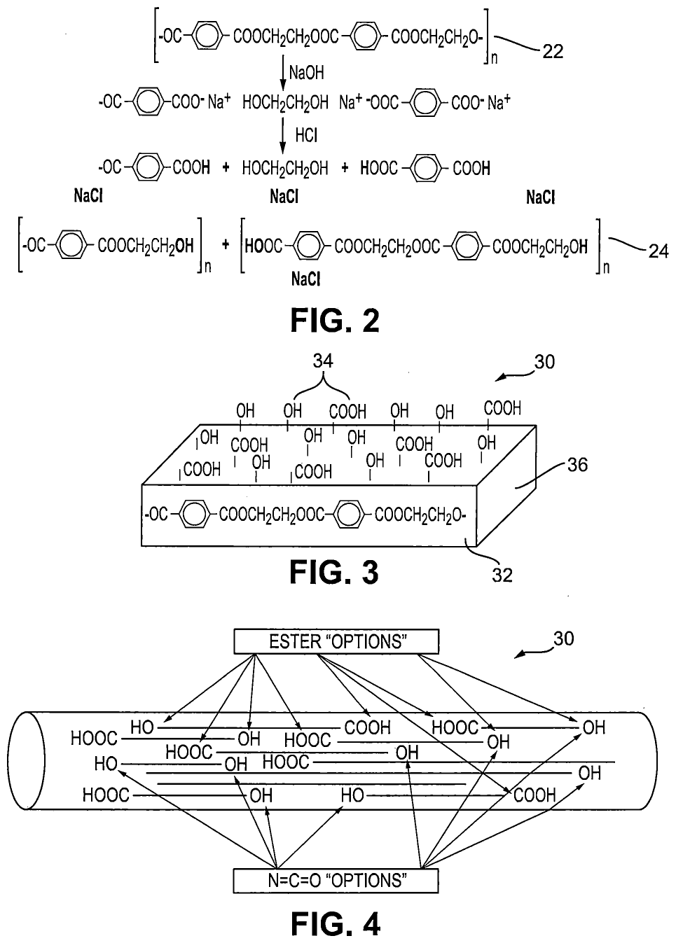

In exemplary embodiments, a process includes modifying a polyester textile surface by soaking or bath exposure to sodium hydroxide (NaOH) to break surface esters and form hydroxy or carboxyl groups, as shown schematically for PET in FIG. 2. Exposure of the PET fiber chain 22 to NaOH base leads to hydrolysis of some of the surface or near-surface esters of the PET and conversion to alcohol and sodium carboxylate groups. Subsequent exposure to hydrochloric acid (HCl) converts the sodium carboxylate groups to carboxylic acids, thereby forming a surface-modified PET fiber chain 24.

Conditions for the NaOH exposure are selected to modify the surface chemistry without changing the bulk properties of the fiber or textile, especially its fiber tenacity. In some embodiments, subsequent treatment with hydrochloric acid (HCl) controls the surface charge to modulate electrostatic interactions and/or to prepare the surface for further chemical modification.

Toluidine blue (TBO) is a stain that binds surface carboxyl groups. In some embodiments, a TBO assay qualitatively determines the degree of surface modification of a polyester. In some embodiments, surface modification conditions are selected to provide a high TBO assay absorbance without detectable fiber deterioration in the form of pitting of the surface of the fiber.

In exemplary embodiments, the conditions of the process provide conversion of the surface functionality for adhesion without reducing the fiber tenacity. In some embodiments, a surface modification process includes exposing the polyester fibers to NaOH in water at a concentration in the range of 0.2 M to 2 M, alternatively 0.3 M to 1.5 M, alternatively about 0.5 M to about 1 M, or any value, range, or sub-range therebetween for at least 30 minutes, alternatively 1 to 30 hours, alternatively 3 to 30 hours, alternatively 6 to 30 hours, alternatively 12 to 24 hours, or any value, range, or sub-range therebetween at a temperature in the range of 10° C. to 60° C., alternatively room temperature (about 20° C.) to 50° C., or any value, range, or sub-range therebetween, followed by rinsing the fibers with HCl in water at a concentration in the range of 0.05 M to 0.2 M, 0.08 M to 0.15 M, about 0.1 M, or any value, range, or sub-range therebetween. In some embodiments, the NaOH in water also includes KMnO₄ at a concentration in the range of 0.05 M to 0.2 M, 0.08 M to 0.15 M, about 0.1 M or any value, range, or sub-range therebetween.

FIG. 3 schematically shows a surface-modified PET article 30 with bulk PET 32 and surface hydroxy or carboxyl groups 34. An end of the surface-modified PET article 30 may form or extend from a fabric flap 36.

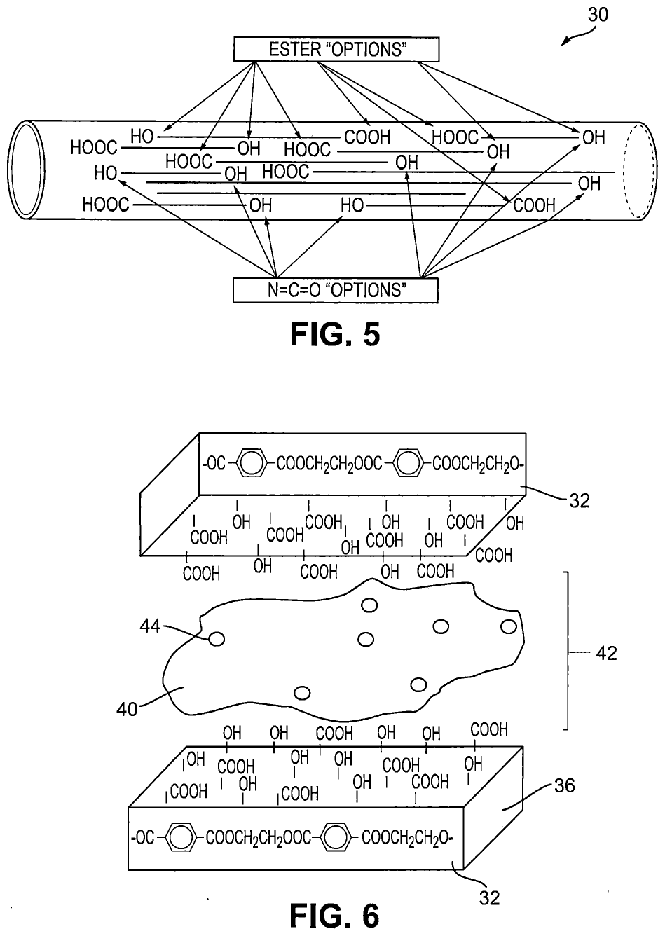

FIG. 4 and FIG. 5 schematically show a surface-modified PET article 30 as including a sacrificial sheath including surface hydroxy and surface carboxyl functional groups around a polymer core of PET.

As shown in FIG. 4, the surface hydroxy groups of the PET article 30 may serve as crosslinking sites for reaction with an isocyanate crosslinker, and both the surface hydroxy and surface carboxyl functional groups may serve as ester coupling sites.

As shown in FIG. 5, the surface hydroxy groups of the PET article 30 may serve as crosslinking sites for reaction with an isocyanate crosslinker, and both the surface hydroxy and surface carboxyl functional groups may serve as ester adhesion anchoring coupling sites.

In exemplary embodiments, a fabrication process prepares all interfacing surfaces for an appropriate chemical bonding routine. In exemplary embodiments, the entire stent-graft process is constructed or built with modified materials, in contrast to conventional processes that are dependent on ligation.

In exemplary embodiments, utilization of the bonding or fusion of materials reduces or eliminates atraumatic edges of ligatures. In exemplary embodiments, utilization of chemistry to fix and construct the three-dimensional structure eliminates the manual labor associated with ligation. These features provide both a biological and an adhesion advantage, as well as an economic benefit.

In exemplary embodiments, a passivation layer binds to the surface of Nitinol and passivates the Nitinol. In some embodiments, the passivation layer also binds to the surface-modified polyester to fuse the Nitinol to the PET. In other embodiments, a tie layer binds the passivation layer and the surface-modified polyester.

In exemplary embodiments, the process includes covalently crosslinking a surface treatment-modified surface of polyester via covalent polycondensation or isocyanate crosslinking. In exemplary embodiments, the passivation layer and/or the tie layer includes a polymer of glycerol and sebacic acid (PGS). The PGS may be formed by a polycondensation reaction including glycerol and sebacic acid, as shown in Formula (2):

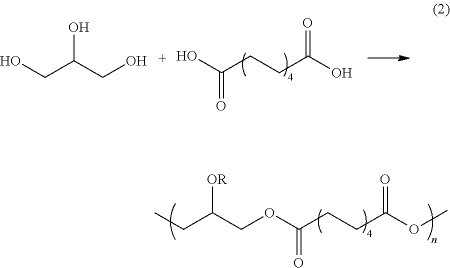

As used herein, “polymer of glycerol and sebacic acid” (PGS) refers to any copolymer including monomer units of glycerol and sebacic acid. The structures of glycerol and sebacic acid are shown in Formula (3):

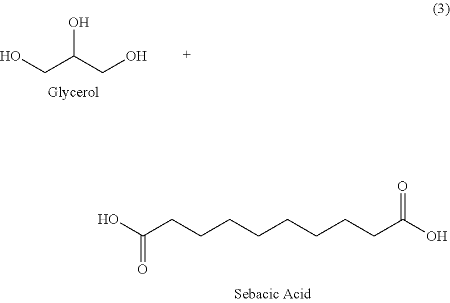

In some embodiments, the PGS polymerization is via a water-mediated polycondensation reaction, such as described in U.S. Pat. No. 9,359,472, which is hereby incorporated by reference herein, that copolymerizes glycerol and sebacic acid.

Although described primarily herein with respect to PGS, it will be appreciated that any polymer formed by a polycondensation reaction from a multi-functional acid monomer and a polyol monomer may be employed as the passivation layer, tie layer, or catechol-modified polymer. Accordingly, in certain aspects of the invention, the polymer is a condensation reaction product of glycerol or other alcohol monomer and a diacid having the formula [HOOC(CH₂)ₙCOOH], where n=1-30, including malonic acid, succinic acid, glutaric acid, adipic acid, pimelic acid, suberic acid, azelaic acid, and itaconic acid, as well as sebacic acid.

Appropriate polyol monomers may include, but are not limited to, glycerol, low molecular weight polyethylene glycol (PEG) (M_{w} about 2000 Da or less), polyvinyl alcohol, xylitol, mannitol, sorbitol, maltitol, erythritol, or isomalt. When more than one polyol monomer is copolymerized with polycarboxylic acid monomer, they may be included at any molar ratio in the range of 1:99 to 50:50.

In some embodiments, the PGS binds directly to the surface-modified polyester by forming ester bonds at the surface. In such embodiments, the PGS prepolymer has available hydroxy and carboxyl groups as crosslinkable functional groups to contribute toward formation of such ester bonds.

In other embodiments, the tie layer includes PGS and an isocyanate crosslinker to crosslink the PGS to a urethane of PGS (PGSU) and to attach the PGS to the surface-modified polyester. Isocyanate can react with available hydroxy groups on either or both the PGS and/or surface-modified polyester.

As shown schematically in FIG. 6, the PGS 40 of the passivation layer or tie layer 42 may include a blocked isocyanate 44, such as, for example, hexamethylene diisocyanate (HMDI), microencapsulated in thermoplastic PGSU microspheres, to provide a thermally-activated adhesive. In such embodiments, the process may be similar to a process of fabric thermal fusion using a heat-activated bonding media.

As noted above, a significant issue with Nitinol implants is Ni leaching. In exemplary embodiments, a process passivates such leachables. PGS and its associated glycerol esters lack the ability to aggressively adhere to Nitinol surfaces. The neat poly(glycerol sebacate) pre-polymer and crosslinked polymer have limited extended interfacial bonding mechanisms. In exemplary embodiments, a passivation agent is coupled to or associated with the PGS of the passivation layer to attach to the Nitinol surface. In exemplary embodiments, the passivation agent both couples the PGS to the Nitinol and passivates the Nitinol.

In exemplary embodiments, the passivating agent is a catechol group. In some embodiments, the PGS is modified to include at least one catechol group for binding to Nitinol and sequestering nickel, thereby allowing the PGS to attach to both the surface-modified PET and the Nitinol. In some embodiments, other passivating agents, such as, for example, surface active agents as anchored additives or free treatments, such as surfactants, wetting agents coupling agents, chelating agents, anti-static agents that may promote covalent passivation, electrostatic passivation, or neutralization of interface or interphase, may be employed. In some embodiments, these surface active agents are anionic, cationic, amphoteric, or non-ionic.

A catechol is a chemical functional group that is a natural metal chelator and is one of the most efficient metal-binding organic groups. A catechol has the chemical structure of Formula (4):

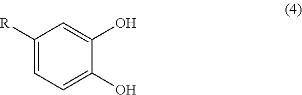

R in Formula (4) may be any chemical structure that includes a functional group that can be coupled or converted to be coupled either directly or indirectly to PGS, such as, for example, a carboxylic acid group. The catechol is preferably a non-toxic natural catechol. In some embodiments, the catechol is 3,4-dihydroxy-9,10-secoandrosta-1,3,5 (10)-triene-9,17-dione, catechin, piceatannol, urushiol, a catecholamine, or quercetin.

In some embodiments, R includes an alkyl chain with a carboxylic acid group. The number of methylene groups in the chain may be varied to vary the linker length of the catechol. In some embodiments, the R group further includes a cleavable bond to provide a release mechanism for the catechol for adhesive applications. Appropriate cleavable bonds may include, but are not limited to, esters, disulfides, or photolabile groups that cleave upon appropriate radiation exposure.

The catechol chemical group is utilized by crustaceans, such as barnacles, and mollusks, such as mussels, to form superior bonding agents. Catechol groups may be used in medical adhesives (see, for example, Park et al., “Advances in medical adhesives inspired by aquatic organisms’ adhesion”, Biomaterials Research, Vol. 21, Art. 16, (2017), incorporated by reference herein).

In exemplary embodiments, a sutureless graft includes a catechol-modified PGS. In exemplary embodiments, the catechol-modified PGS is derived from all natural materials. In exemplary embodiments, the natural materials are all plant-based. In exemplary embodiments, the PGS structure is selected or modified for tissue compliance.

A catechol-modified PGS has many advantages over the catechol-modified polysaccharides and proteins reviewed by Costa. PGS is elastomeric and in exemplary embodiments is tailored to have an elastic modulus that approximates or matches the elastic modulus of the tissue or other substrate being bonded to provide a catechol-containing tissue-substrate compliance-matched adhesive bond line or tie layer at the mechanobiologic interface rather than a stiff structural adhesive. This makes the adhesive bulk property more physiological-like. Without the elasticity, a bond line may be fractured with the movement involved at many types of implantation sites. The common plastic lactides and glycolides lack the hydroxyl functionality on the backbone to attach a catechol.

PGS is a surface-eroding polymer that does not osmotically draw in water and thus shows minimal swelling in water and therefore is more hydrolytically stable and avoids some of the mechanical limitations of polysaccharides and proteins.

PGS is bioresorbable and breaks down into useful metabolic components rather than the non-bioresorbable waste products from degradation of the polymers described by Costa. This bioresorption promotes scaffolding regeneration. In exemplary embodiments, the catechol-modified PGS is an elastomeric, metabolic monomer component, surface eroding, biodegradable, non-immunogenic, antithrombic, regenerative scaffold polymer, where the adhesive functionality at the site of the repair provides immediate “biologic” fixation followed by composite support of regeneration with the eventual bioresorption and metabolic feeding of the repairing cells.

PGS has antimicrobial properties that inhibit infection at the implant site. The antimicrobial properties of the PGS are enhanced by the catechol and may prevent an overexposure of regenerative tissues to the catechol.

In some embodiments, the R group of the structure of Formula (4) is —CH₂CH(NH₂)COOH such that the monomer unit containing the catechol group is L-dopamine, which has the structure of Formula (5):

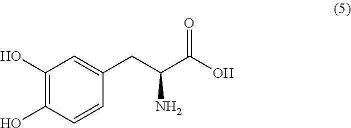

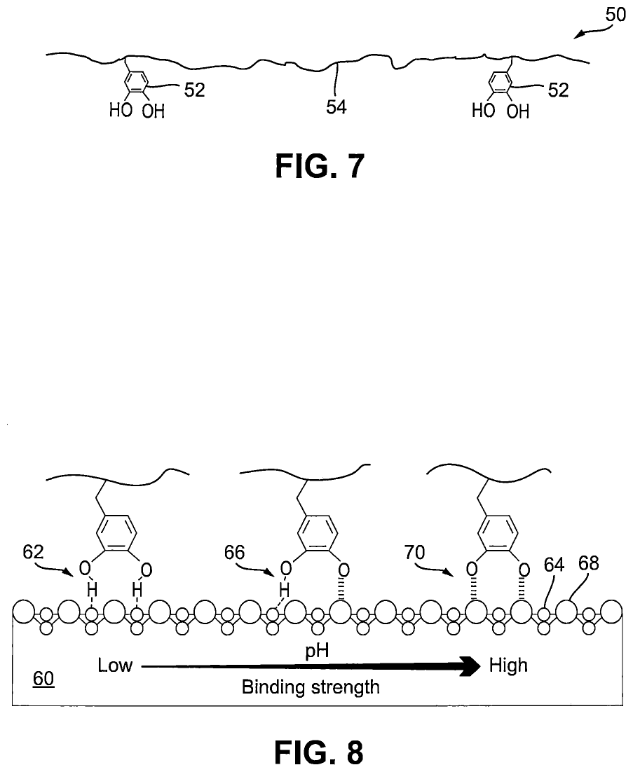

In some embodiments, the R group of the structure of Formula (4) is CH₂(CH₂)ₙCOOH, where n=1 to 10 and the catechol-containing monomers are added at various times to the polycondensation reaction between the glycerol and sebacic acid to form the catechol-modified PGS with the catechol-containing monomers being tethered to the backbone of the polymer. FIG. 7 schematically shows the resulting structure of the catechol-modified PGS 50, with the catechol groups 52 being pendant from the PGS backbone 54.

In other embodiments, the catechol-containing unit is reacted in a pre-monomer reaction, such as, for example, with an organic diacid, to offer an option to post-modify a pre-polymer or provide an additive option to a glycerol ester design.

In other embodiments, the catechol-containing modification is attached as a pendant group to the PGS after formation of the PGS. For example, the catechol-containing modification may include a functional group, such as, for example, a carboxylic acid, an alcohol, or an amine, to couple to an activated carboxylate or hydroxy group of the glycerol in the PGS to form a catechol-containing PGS, with the catechol group 52 being pendant. The coupling can be performed with a carbodiimide, such as, for example, 1-ethyl-3-(3-dimethylaminopropyl)carbodiimide (EDC), N,N′-diisopropylcarbodiimide (DIC), or N,N′-dicyclohexylcarbodiimide (DCC). Additionally, the catechol modification can be reacted onto the polymer backbone using 1,1′-carbonyldiimidazole (CDI).

In some embodiments, the catechol-containing PGS is a catechol-containing PGSU. In some embodiments, a catechol-containing PGS is modified to a catechol-containing PGSU. In other embodiments, PGS is modified to PGSU and the catechol-containing modification is attached as a pendant group to the PGSU polymer after formation of the PGSU polymer. In exemplary embodiments, the crosslinking occurs under conditions that promote crosslinking of the PGS hydroxy groups over crosslinking of the catechol hydroxy groups.

A catechol-modified PGS provides a biodegradable elastomer with adhesive or surface-coordinating properties more biomimetic than surfactants or conventional coupling agent chemistries for human implant applications. In some embodiments, the biodegradability of the PGS allows for tissue integration into the graft, leading to tissue fixation of the graft and better physiological compliance to natural tissue. In exemplary embodiments, the catechol-modified PGS acts as a non-immunogenic passivation agent that further prevents hypersensitivity while acting as a functional featured additive. In some embodiments, a post-polymerization process in the presence of the catechol groups leads to a higher degree of crosslinking based on the participation of catechol hydroxy groups in the crosslinking.

In exemplary embodiments, the catechol groups of a catechol-modified polymer of glycerol and sebacic acid chelate and/or coordinate nickel at the surface of Nitinol to both sequester the nickel from leaching and prevent tissue interaction with the nickel. Additionally, the surface passivated by the catechol-modified PGS serves as a primer coating for subsequent applications of PGS resin as either a coating or adhesive. Thus, the catechol-modified PGS may serve as both a passivation coating and a tie coating.

FIG. 8 schematically shows three different ways in which a catechol 54 can interact with a titanium dioxide 60 surface, such as present on the outer surface of a Nitinol article, in the order of increasing bond strength. The weakest bonding strength 62 is with both catechol hydroxy groups forming hydrogen bonds with surface oxygen atoms 64. A stronger bonding strength 66 is with one catechol hydroxy group forming a hydrogen bond with a surface oxygen atom 62 and the other forming a coordination bond with a surface titanium atom 68. The strongest bonding strength 70 is with both catechol hydroxy groups forming coordination bonds with surface titanium atoms 68.

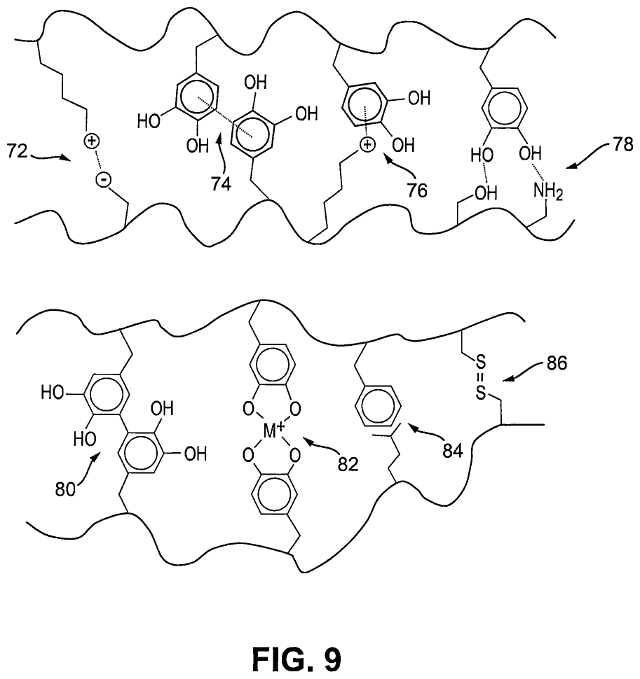

Poly(glycerol sebacate) and its associated glycerol esters lack the ability to aggressively adhere to Nitinol surfaces. The neat PGS pre-polymer and crosslinked polymer have limited extended interfacial bonding mechanisms. Incorporation of the catechol functionality provides a plurality of surface features to PGS. These features may include, but are not limited to, as shown schematically in FIG. 9, electrostatic interactions 72, π-π interactions 74, cation-π interactions 76, additional hydrogen-bonding potential 78, covalent crosslinking potential 80, metal coordination potential 82, hydrophobic interactions 84, and disulfide bond potential 86. The consequence is a multifunctional polymer with multiple bonding options.

There are few monomers, additives, or adjunct materials that support non-immunogenic existence in an implantable structure such as one including Nitinol wire. Embodiments disclosed herein maintain a theme of non-immunogenic polymer technology, as the catechol adhesive is a bioadhesive.

Coupling catechol to PGS provides the associated elastomeric property of PGS at the interface. This establishes a bonding feature that can manage interfacial stress, because PGS elastomer creates a rugged bonding capability, in contrast to rigid polymeric bond lines that are prone to force fractures. The elastomeric feature absorbs both vibrational and impact forces. This is a contributing feature that supports its application in a sutureless stent graft construct.

A catechol-modified PGS may, for example, fix textiles to textiles, textiles to metallics, and/or textiles to plastics and other elastomers in forms, such as, for example, coatings, adhesives, passivation treatments, composites, and other bonding interfaces based on the selection of catechol-to-subject interaction. As noted above, the bonding specificity of the catechol to a substrate may be achieved by any of a number of different mechanisms.

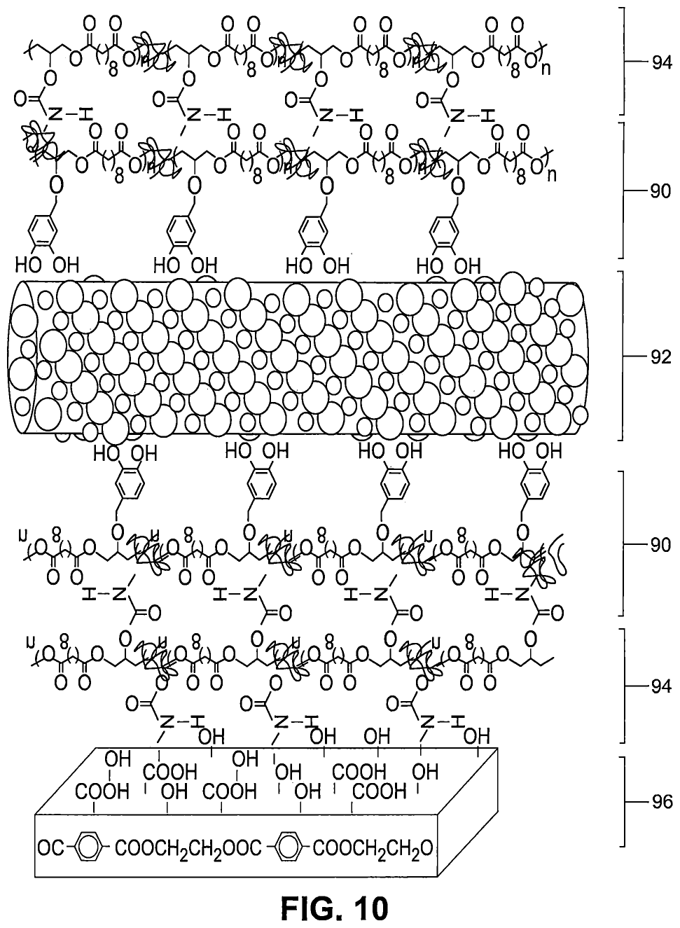

In some embodiments, the catechol-modified PGS serves as a passivation layer as the first interface upon which a sutureless implantable device is formed, as shown schematically in FIG. 10. First, the passivation layer 90 of catechol-modified PGS binds by way of the catechol groups to the surface of the Nitinol wire 92, forming the structural support for the device. Then, a crosslinkable polymer, such as, for example, PGSU, forms a tie layer 94 between the catechol-modified PGS coating and a PET or other polyester layer or textile 96.

In exemplary embodiments, the passivation layer 90 is a thin layer coating the Nitinol wire 92, with the bulk polymer properties coming primarily from the tie layer 94, which does not include catechol, since the catechol is desirable primarily to act at the Nitinol surface and excess unbound catechol may be toxic or otherwise detrimental to the implantation environment.

Being biodegradable, the PGS eventually degrades based on the degree of crosslinking. Nitinol leaching may occur throughout the lifetime of the implant. A significant amount of work has been done on the actual processing of the Nitinol to address this issue. A benefit of the catechol-modified PGS coating is to minimize nickel exposure during the initial inflammatory response as a result of the surgical trauma and subsequent healing process. Thus, the biocompatibility of the PGS plays an important role initially after implantation.

In other embodiments, the catechol promotes adhesion of a catechol-modified polymer of glycerol and sebacic acid to metal complexing surfaces, such as orthopedic devices and structural heart valves, to insure early stage control of the immune response.

In other embodiments, the catechol sequesters selected matrix metalloproteases (MMPs) to reduce or prevent their adverse influence in diabetic wound care.

Other applications for a catechol-modified polymer of glycerol and sebacic acid may include, but are not limited to, biodegradable pressure-sensitive adhesives for environmental use, surgical adhesives for bone and/or soft tissue, tissue scaffolding for bone to promote osteoconduction and support osteoinductive growth promoters, water remediation for trapping of toxic ions and/or for controlled release of biocidal metals, such as, silver, copper, and/or zinc, textile coatings for filtration including vapor deposition on flow-through filters for ion extraction, surface treatment of bioreactor walls for ion sequestering and/or ion controlled release, and/or polymeric stock for chromatographic microparticle development.

In some embodiments, the polyester reactive vehicle coating and adhesives permit construction of a stent graft composite with limited or no ligation to fix polyester to the Nitinol scaffold.

The materials of construction for the polyester and Nitinol may be derived from standard PET textiles and standard Nitinol scaffolding. In some embodiments, however, the Nitinol surface topography and chemical composition may be customized specifically for enhanced bonding. In some embodiments, for example, the PET fibers and textiles are independently modified by NaOH treatment prior to textile formation. In some embodiments, for example, the Nitinol scaffolding is independently passivated either by a manufacturing process or by treatment with catechol-containing PGS. In other embodiments, a polyester graft textile is designed with loops or flaps to fix or capture Nitinol wire to be converted into a composite.

The PGS may also be designed to include photocurable functional groups, whereby the initial fixation is established with ultraviolet (UV) light or other energy conversion followed by advanced permanent crosslinking with heat or microwave or radiofrequency (RF), for an example.

Other applications for sutureless fixation of polyester to Nitinol and/or catechol-modified PGS may include, but are not limited to, biodegradable adhesive applications, bioremedial applications including both medical and environmental applications, surgical adhesives, and/or antifouling coatings.

In some embodiments, the catechol-modified PGS is part of an orthopedic implant. In some embodiments, the catechol-modified PGS adheres to bone. In some embodiments, the catechol-modified PGS promotes tendon or cartilage regeneration.

In exemplary adhesive applications, the catechol-modified PGS provides a thin but compliant adhesive interface. In some embodiments, the catechol-modified PGS is able to extend past the interface and into the substrate to form an interpenetrating adhesive interphase. In some embodiments, an adhesive includes the catechol-modified PGS as a thin layer on a surface of a bulk substrate of PGS with significantly less catechol than in the thin layer. In some embodiments, the bulk substrate PGS is free or substantially free of catechol. In some embodiments, the catechol-modified PGS permits an adhesive bond with a thin but resilient bond line.

In some embodiments, the adhesive containing a catechol-modified PGS is reversibly adhesive based on the chelating behavior of the catechol.

In some embodiments, the catechol-modified PGS forms an antifouling coating on a metal surface, such as, for example, the water-contacting metallic surfaces of a ship. In some embodiments, the catechol-modified PGS is a catechol-modified PGSU. In some embodiments, the catechol-modified PGS serves as both a coating and a controlled biocide release vehicle.

### EXAMPLES

The invention is further described in the context of the following examples which are presented by way of illustration, not of limitation.

### Example 1

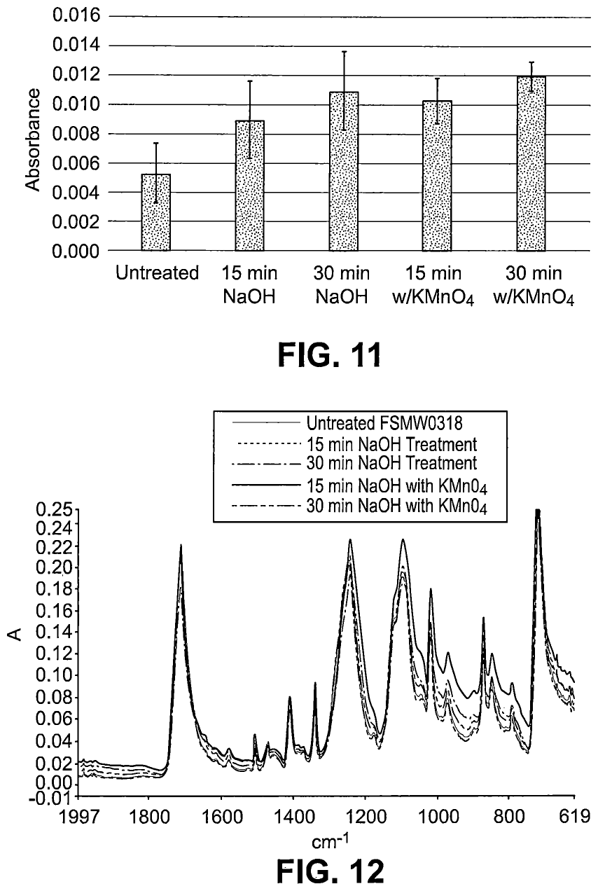

Five different treatment conditions were applied to PET textiles to chemically modify the surface to include surface hydroxy and/or carboxyl groups and the treated textiles were then tested by a TBO assay to determine the relative amount of carboxylic acid groups at the surface of the treated textile. A first PET textile received no treatment and served as a control. A second PET textile was exposed to a solution of 1 M NaOH at 50° C. for 15 minutes. A third PET textile was exposed to 1 M NaOH at 50° C. for 30 minutes. A fourth PET textile was exposed to a solution of 1 M NaOH and 0.1 M KMnO₄ at 50° C. for 15 minutes. A fifth PET textile was exposed to a solution of 1 M NaOH and 0.25 M KMnO₄ at 50° C. for 20 minutes. FIG. 11 shows the assay results, with higher absorbance indicating higher levels of surface carboxylic acid groups. FIG. 12 shows the Fourier-transform infrared (FTIR) spectroscopy data for the treated surfaces.

FIG. 11 shows that the treatments increased the number of surface carboxylic acid groups, with the longer treatment times producing more surface carboxylic acid groups. These results at 50° C. and longer times were better than at 100° C. for shorter amounts of time (data not shown), indicating that longer times at lower temperatures were more effective. The presence of KMnO₄ seemed to influence the FTIR results but not the TBO assay results.

### Example 2

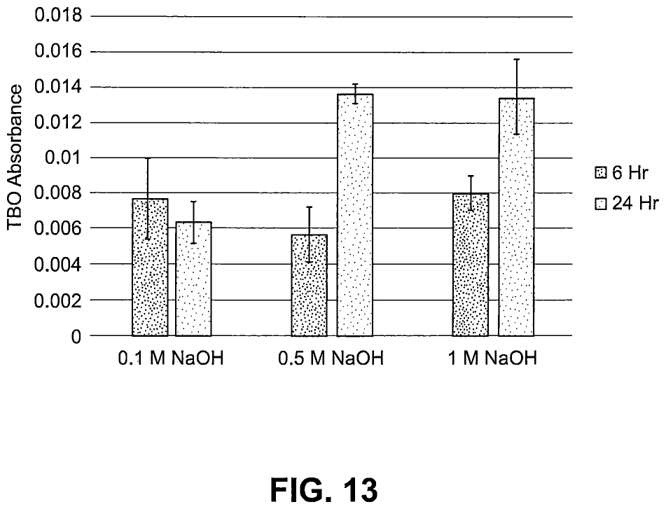

PET textiles were treated under different NaOH concentrations for different amounts of time at room temperature to determine the relative amounts of chemical modification to the surface to include surface hydroxy and/or carboxyl groups. Six different treatments were tested: 0.1 M NaOH for 6 hours, 0.1 M NaOH for 24 hours, 0.5 M NaOH for 6 hours, 0.5 M NaOH for 24 hours, 1 M NaOH for 6 hours, and 1 M NaOH for 24 hours. FIG. 13 shows the TBO assay results, with higher absorbance indicating higher levels of surface carboxylic acid groups.

The 0.5 M and 1 M concentration treatments for 24 hours provided similar results to each other and much better results than the other for conditions tested.

All above-mentioned references are hereby incorporated by reference herein.

While the invention has been described with reference to one or more exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention but that the invention will include all embodiments falling within the scope of the appended claims. In addition, all numerical values identified in the detailed description shall be interpreted as though the precise and approximate values are both expressly identified.

What is claimed is:

1. An implantable device comprising a first polymer of glycerol and sebacic acid, the first polymer of glycerol and sebacic acid comprising a catechol group.

2. The implantable device of claim 1 further comprising Nitinol.

3. The implantable device of claim 2, wherein the first polymer of glycerol and sebacic acid is coated on the Nitinol.

4. The implantable device of claim 3, wherein the catechol group is bound to the surface of the Nitinol, thereby passivating the Nitinol.

5. The implantable device of claim 1, wherein the implantable device is a sutureless graft.

6. The implantable device of claim 1 further comprising a Nitinol wire, a passivation layer comprising the first polymer of glycerol and sebacic acid, a surface-modified polyester textile, and a tie layer, wherein the passivation layer is bound to the Nitinol wire, the tie layer binds the surface-modified polyester textile to the passivation layer such that the surface-modified polyester textile is suturelessly fixed to the Nitinol wire, and the surface-modified polyester textile comprises a textile of a polyester having a modified surface that provides surface crosslinkable groups.

7. The implantable device of claim 6, wherein the polyester is selected from the group consisting of polyethylene terephthalate (PET), polylactic acid (PLA), polyglycolic acid (PGA), poly(lactic-co-glycolic acid) (PLGA), polycaprolactone (PCL), and polyhydroxyalkanoate (PHA).

8. The implantable device of claim 6, wherein the catechol groups suturelessly fix the first polymer of glycerol and sebacic acid of the passivation layer to the surface of the Nitinol wire.

9. The implantable device of claim 6, wherein the tie layer comprises a second polymer of glycerol and sebacic acid bound to the surface-modified polyester textile by ester bonds.

10. The implantable device of claim 6, wherein the tie layer comprises a urethane-crosslinked polymer of glycerol and sebacic acid bound to the surface-modified polyester textile and to the passivation layer by urethane bonds.

11. The implantable device of claim 1, wherein the first polymer of glycerol and sebacic acid is elastomeric, non-immunogenic, and resorbable, has a chemical structure selected to match a compliance of a tissue or other substrate, and acts as an adhesive bond-line or tie layer for the implantable device.

12. A composition comprising a polymer of glycerol and sebacic acid, the polymer of glycerol and sebacic acid comprising a catechol group.

13. A process comprising suturelessly fixing a surface-modified polyester textile to Nitinol wire, wherein the surface-modified polyester textile comprises a textile of a polyester having a modified surface that provides surface crosslinkable groups.

14. The process of claim 13, wherein the polyester is selected from the group consisting of polyethylene terephthalate (PET), polylactic acid (PLA), polyglycolic acid (PGA), poly(lactic-co-glycolic acid) (PLGA), polycaprolactone (PCL), and polyhydroxyalkanoate (PHA).

15. The process of claim 14, wherein the suturelessly fixing comprises modifying an original surface of the textile of the polyester to form the surface-modified polyester textile, wherein the surface crosslinkable groups include surface hydroxy groups and surface carboxyl groups.

16. The process of claim 15, wherein the modifying comprises surface treating the original surface of the textile of the polyester by exposing the original surface to sodium hydroxide and hydrochloric acid to break surface ester groups and form the surface hydroxy and carboxyl groups.

17. The process of claim 15 further comprising coupling a first polymer of glycerol and sebacic acid to the surface-modified polyester textile.

18. The process of claim 17, wherein the coupling comprises crosslinking the first polymer of glycerol and sebacic acid to the surface hydroxy groups of the surface-modified polyester textile by a diisocyanate crosslinker.

19. The process of claim 17, wherein the suturelessly fixing comprises passivating the Nitinol wire with a passivation layer comprising a second polymer of glycerol and sebacic acid comprising catechol groups.

20. The process of claim 19, wherein the catechol groups bind the second polymer of glycerol and sebacic acid to the Nitinol wire.

21. The process of claim 20, wherein the first polymer of glycerol and sebacic acid serves as a tie layer between the passivation layer and the surface-modified polyester textile.
